(19) United States
(12) Patent Application Publication
(10) Pub. No.: US 2025/0362592 A1
(43) Pub. Date: Nov. 27, 2025

(54) LIVE CELL TATTOOS

(71) Applicant: The Johns Hopkins University, Baltimore, MD (US)

(72) Inventors: David H. GRACIAS, Baltimore, MD (US); Kam Sang KWOK, Ellicott City, MD (US); Luo GU, Baltimore, MD (US); Gayatri PAHAPALE, Baltimore, MD (US); Soo Jin CHOI, Baltimore, MD (US); Yi ZUO, Baltimore, MD (US)

(21) Appl. No.: 19/216,809

(22) Filed: May 23, 2025

Related U.S. Application Data
(60) Provisional application No. 63/650,971, filed on May 23, 2024.

(51) Int. Cl. G03F 7/00; C12N 1/20; C12N 5/071

(52) U.S. Cl. CPC G03F 7/0002; C12N 1/20; C12N 5/0602

(57) ABSTRACT

A hybrid micro and nanotransfer printing process that can bond lithographically-defined nanopatterns to live cells, tissue, organs, or microorganisms under physiological conditions is described. Advantageously, the process is flexible, the nanopatterns and the live cells maintain structural integrity, the nanopatterns are compatible with cell culture media, and the nanopatterns display appropriate adhesion to the live cells and other living entities.

### CROSS-REFERENCE TO RELATED APPLICATIONS

This application claims the benefit of U.S. Provisional Application No. 63/650,971, filed May 23, 2024, the contents of which is incorporated herein by reference in its entirety.

### FEDERALLY SPONSORED RESEARCH OR DEVELOPMENT

This invention was made with government support under grant 21RT0264-FA9550-21-1-0284 awarded by the Air Force Office of Scientific Research, grant R03AG073834 awarded by the National Institutes of Health, grant EFMA-1830893 awarded by the National Science Foundation, and grant CMMI 1635443 awarded by the National Science Foundation. The government has certain rights in the invention.

### BACKGROUND

Engineers have long sought to merge nanoelectronics, nanophotonics, and stimuli-responsive materials with the human body across length scales of organs to single cells. To create smart devices tailored to the soft, dynamic, and three-dimensional (3D) surfaces of biological systems, it is necessary to establish methods that can reliably integrate well-defined nanopatterns such as electrode arrays, antennas, and circuits onto living cells and tissues. In the last few decades, advances in very large-scale integration (VLSI) and microelectromechanical systems (MEMS) have enabled the fabrication of sophisticated devices like transistors, integrated circuits, and sensors with exquisite nanoscale resolution. More recently, the assembly of materials and devices on flexible substrates that can mold to curvilinear surfaces has been achieved via laser printing, 3D printing, micro pick-and-place systems, and self-assembly. These top-down processes, however, often utilize harsh chemicals, high temperatures, or vacuum techniques that are incompatible with living cells, tissues, and soft aqueous materials.

To address this challenge, researchers have explored alternative approaches to creating biological interfaces, such as depositing force-mediating nanoparticles on cells or 3D bioprinting composite formulations of nanomaterials and cells. However, these biocompatible techniques often possess limited throughput and resolution, especially at submicrometer length scales. Yet others have shown that living cells can internalize microstructures, such as radio frequency identification (RFID), force and pressure sensors, barcodes, magnetic antennas, and microrobots. These studies demonstrate the possibility of interfacing various materials with live cells and tissues. Lithographic nanopatterning techniques such as photolithography, electron-beam lithography, and nanoimprint lithography (NIL) have revolutionized modern-day electronics and optics. Yet, their application for creating nanobio interfaces is limited by the cytotoxic and two-dimensional nature of conventional fabrication methods. Accordingly, a lithography-based technique for systematically integrating nanomaterials onto live cells with a high spatial resolution and yield has yet to be realized.

Nanotransfer printing (nTP) offers a high-throughput approach to printing large-area arrays of nanopatterns on unconventional 3D substrates [Carlson et al., 2012], such as polymers [Jeong et al., 2014], elastomers [Ahn et al., 2023], and hydrogels [Ko et al., 2021]. For instance, Jeong et al. [Jeong et al., 2016] used a solvent-assisted nTP technique to print arrays of plasmonic silver nanowires on soft contact lenses for enhanced Raman signals, which enabled glucose detection at low concentrations. Similarly, Ko et al. printed Au nanowires on hyaluronic acid film to develop smart contact lenses capable of treating Irlen syndrome [Ko et al., 2021]. While these nTP techniques are capable of printing large-area nanopatterns on flexible substrates in parallel, they require organic solvents (e.g., toluene, acetone), high pressure (e.g., 3 bar), or high temperatures (e.g., 45-100° C.), all of which are highly unfavorable conditions for living systems.

There continues to be a need for a technique for integrating nanomaterials onto live cells and other non-rigid materials, including living and soft biological materials, with a high spatial resolution and yield. Towards that end, a hybrid nTP process that can bond lithographically-defined micro-and nanopatterns to live cells, tissue, and microorganisms under physiological conditions is described.

### SUMMARY

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

In some aspects, the present disclosure relates to a method of hybrid micro or nanotransfer printing (nTP) a nanopattern onto an article, said method comprising: preparing the nanopattern comprising at least one nanopattern material on a first substrate;
transferring the nanopattern material onto a second substrate;
functionalizing the nanopattern material on the second substrate with at least one functionalizing compound;
casting the functionalized nanopattern material with at least one casting material to effectuate delamination of the nanopattern material from the second substrate;
chemically conjugating the nanopattern material with at least one conjugation compound to assist transfer of the nanopattern material onto the article;
positioning the chemically conjugated nanopattern material on the article; and
dissociating of the at least one casting material with a dissociation composition to yield the article comprising the nanopattern. In some embodiments, the article comprises at least one of cells, tissues, organs, and microorganisms. In some embodiments, the article is living.

In another aspect, an article comprising a nanopattern positioned thereon is described. In some embodiments, the article comprises at least one of cells, tissues, organs, and microorganisms.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1. Schematic illustration showing the step-by-step fabrication of the Au NIL arrays on Si wafers and subsequent transfer onto glass coverslips and cell sheets. The main steps include (a) spin-coating the sacrificial layer (PMGI) on a Si wafer, (b) spin-coating the NIL resist (mr-I 7030), (c) thermal NIL using a Si stamp, (d) de-scumming the residual NIL resist with oxygen plasma, (e) thermally evaporating 5 nm of Cr and 50 nm of Au, (f) lifting off the NIL resist in acetone, (g) spin-coating the carrier film (PMMA), (h) dissolving the sacrificial layer in positive photoresist developer (MF-26A) and Cr in Cr S6 etchant (Cr Cermet Etchant TFE) followed by rinsing with water, (i) manually picking up the Au NIL-array from the water-air interface using a glass coverslip, (j) removing the carrier film using an oxygen plasma, (k) functionalizing the Au NIL-array with cysteamine, 1) casting alginate hydrogel on the Au NIL-array, m) peeling off the Au Nil-array printed alginate hydrogel from the glass coverslip and placing it pattern-side up, n) bioconjugating gelatin to the Au NIL-array according to the process detailed in FIG. S4, o) seeding NIH/3T3-GFP cells on the Au NIL-array printed hydrogel and culturing for 24 hours, p) flipping over the cell-seeded hydrogel onto a gelatin-coated glass coverslip, and q) dissociating the alginate hydrogel with 20 mM EDTA to obtain the Au NIL-array printed cells.

FIG. 2. Characterization of the Au NIL-arrays on alginate hydrogels. 2A: Optical images of the Au NIL-arrays functionalized with 3-mercaptopropionic acid and ethanol (top), ethanol (middle), or cysteamine and ethanol (bottom). 2B: Transfer yield of the Au NIL-arrays functionalized with different molecules from the glass coverslips to the alginate hydrogels. C: Proposed mechanism for enhancing the transfer yield of the Au NIL-arrays to the alginate hydrogel with positively charged cysteamine.

FIG. 3. Schematic illustration and microscopy images showing the Au NIL-array's transfer process to the alginate hydrogel. 3A: Steps for the Au NIL-array transfer to alginate hydrogel. (i) the Au NIL-array is transferred to a glass coverslip and then functionalized the Au with cysteamine. (ii) the alginate hydrogel transfer layer is cast on top of the Au NIL-array. (iii) the alginate hydrogel containing the Au NIL-array was peeled off and placed it pattern-side up. 3B: Optical image of the Au NIL-array printed alginate hydrogel. 3C: Top view SEM images of the Au NIL-dot printed alginate hydrogel transfer layers. 3D: Top view SEM images of the Au NIL-wire printed alginate hydrogel transfer layers. Scale bars represent the same lengths in panels 3C and 3D.

FIG. 4. Schematic illustration showing the steps for the bioconjugation of gelatin to the Au NIL-arrays.

FIG. 5. Characterization of NIH/3T3-GFP fibroblast migration on the Au NIL-array printed alginate hydrogels. 5A: Time-lapse confocal phase images of a representative NIH/3T3-GFP cell on the Au NIL-dot printed alginate hydrogel show changing migration trajectory. 5B: Time-lapse confocal phase images of a representative NIH/3T3-GFP cell on the Au NIL-wire printed alginate hydrogel show linear migration trajectory. 5C: Angular distribution plot of the cell migration orientation and distance. The plot shows that cells migrated on the Au NIL-dots (blue) with no significant directional preference, whereas cells on the Au NIL-wires (red) migrated primarily along the direction of the wires. 5D: Elongation factor of the cells on Au NIL-dots (blue) and Au NIL-wires (red). Cell elongation is more pronounced on Au NIL-wires. Data are presented as mean±SD (n=30 cells). Statistical analysis was performed using the unpaired two-sided t test. ****p<0.0001. 5E: The average cell migration speed is higher on the Au NIL-dots (blue) than on the Au NIL-wires (red). Data are presented as mean±SD (n=30 cells). Statistical analysis was performed using the unpaired two-sided t test. **p<0.01.

FIG. 6. Schematic illustration and microscopy images showing the biotransfer printing process of Au NIL-wires on rat brains. 6A: Steps for the Au NIL-array transfer to a rat brain. (i) First, the Au NIL-array was transferred to a glass coverslip and functionalized the Au surface with cysteamine. (ii) Next, alginate hydrogel was casted on top of the Au NIL-array. (iii) Then, the alginate hydrogel containing the Au NIL-array was peeled off and the patterned surface bioconjugated with gelatin before placing it on top of a rat brain. (iv) Finally, the alginate hydrogel was dissociated with 20 mM EDTA. 6B: Optical images of Au NIL-wires on a rat brain before dissociation of the alginate hydrogel with 20 mM EDTA. 6C: Optical images of Au NIL-wires on a rat brain after dissociation of the alginate hydrogel with 20 mM EDTA. 6D: Magnified images of Au NIL-wires on a rat brain before dissociation of the alginate hydrogel with 20 mM EDTA. 6E: Magnified images of Au NIL-wires on a rat brain after dissociation of the alginate hydrogel with 20 mM EDTA. 6F: Side view image of Au NIL-wires on a rat brain after dissociation of the alginate hydrogel transfer layer with 20 mM EDTA. The inset shows a magnified view of the Au NIL-wires on the rat brain obtained by a laser scanning microscope.

FIG. 7. Characterization of the biotransfer printed Au NIL-wires on a rat brain slice. 7A: Optical image of the Au NIL-wires on a rat brain slice before dissociating the alginate hydrogel. 7B: Optical image of the Au NIL-wires on a rat brain slice after dissociating the alginate hydrogel with 20 mM EDTA.

FIG. 8. Characterization of the biotransfer printed Au NIL-arrays on NIH/3T3-GFP cell sheets. 8A: Schematic illustration showing the steps for transferring the Au NIL-array to a cell sheet. (i) First, the Au NIL-array was transferred to a glass coverslip and functionalized the Au with cysteamine. (ii) Next, alginate hydrogel was casted on top of the Au NIL-array. (iii) Then the alginate hydrogel containing the Au NIL-array was peeled off and the patterned surface bioconjugated with gelatin. (iv) Then NIH/3T3-GFP cells were seeded on top of the Au NIL-array printed hydrogel. (v) Next, the cell-seeded hydrogel was flipped over onto a gelatin-coated coverslip. (vi) Finally, the alginate hydrogel was dissociated with 20 mM EDTA. 8B: Live-dead assay of NIH/3T3-GFP cells patterned with Au NIL-dots after dissociating alginate hydrogel with 20 mM EDTA. Dead cells in red fluorescence. 8C: Live-dead assay of NIH/3T3-GFP cells patterned with Au NIL-wires after dissociating alginate hydrogel with 20 mM EDTA. Dead cells in red fluorescence. 8D: Viability of the cells with Au NIL-dots or NIL-wires. 8E: Optical image of an 8×8 mm array of Au NIL-arrays printed on an NIH/3T3-GFP cell sheet. 8F: SEM image of Au NIL-dots on fibroblasts. 8G: SEM image of Au NIL-wires on fibroblasts. 8H: Optical image of the microcell patches with Au NIL-dots. 8I: SEM image of a microcell patch with Au NIL-dots. 8J: Magnified SEM image of a cell in the microcell patch with Au NIL-dots.

FIG. 9. SEM images of NIH/3T3-GFP fibroblasts. 9A: SEM image of cells without Au NIL-arrays on a glass coverslip, 9B: SEM image of distorted Au NIL-dots on cells that were cultured for 4 hours, 9C: SEM image of NIH/3T3-GFP fibroblasts covered with a thin, porous film and Au NIL-dots. The cells were cultured for at least 24 hours.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, a “nanopattern” can be in a form of a substantially spherical nanodot, a pyramidal particle, a multi-arm particle, a cubic nanoparticle, a nanotube particle, a nanowire particle, a nanofiber particle, a nanoplate particle, or any combination thereof. In some embodiments, the nanopatterns are arrays. In some embodiments, the nanopatterns include functional patterns such as biosensors, logic and/or memory circuits, bar and/or QR codes or tags or exoskeletons, as understood by the person skilled in the art. In some embodiments, the nanopatterns are capable of recording and transmitting information. The nanopatterns are defined by a nanopattern material comprised of essentially any material. In some embodiments, nanopattern material can comprise, for example, at least one of metals, metal alloys, or can be a material that is substantially crystalline, substantially mono-crystalline, poly-crystalline, amorphous or a combination thereof. In some embodiments, the nanopattern material can comprise a group IV semiconductor compound, a group II-VI semiconductor compound, a group III-V semiconductor compound, a metal or a metal alloy, an insulator, or a high-K material. In some embodiments, the nanopattern material is a molecular, biomolecular, macromolecular or polymer patch. Although the term “nanopattern” will be used hereinafter, it should be appreciated by the person skilled in the art that the pattern printed using the methods described herein can comprise a micropattern, or a combination of a micropattern and a nanopattern.

As used herein, “nanoparticles” can have a characteristic dimension that is less than about 100 μm, for example less than 10 μm, less than 1 μm, or even less than 100 nm. In some embodiments, each of these nanoparticles may have a characteristic dimension less than about 10 μm. In some embodiments, each of these nanoparticles may have a characteristic dimension less than about 1 μm. In some embodiments, each of these nanoparticles may have a characteristic dimension less than about 100 nm. In some embodiments, the nanoparticles comprise nanodots. In some embodiments, the nanoparticles comprise nanowires.

As used herein, “non-rigid” articles can be any material, living or non-living, and they have some amount of flexibility or lack of a defined exterior framework including, but not limited to, prokaryotic, eukaryotic and mammalian cells, fibers, microparticles, hydrogels, 2D materials, tissues, organs, polymers, microorganisms, and any combination thereof.

Broadly, the present disclosure relates to a biocompatible and cost-effective hybrid nTP process for printing patterns/arrays on living cells, tissues, and soft aqueous materials. The hybrid nTP process comprises at least three steps: (1) conventional lithography (e.g., thermal nanoimprint lithography (NIL)) and subsequent transfer onto substrates (e.g., glass coverslips) to obtain a pattern (e.g., arrays of Au nanodots and nanowires), (2) functionalization (.e.g., using an amine) of the patterns followed by casting (e.g., using an alginate hydrogel) to delaminate the patterns from the substrate, and (3) chemical conjugation of the patterns (e.g., with gelatin) to assist transfer onto tissue or living cells followed by the dissociation of the casting material (e.g., alginate hydrogel) with ethylenediaminetetraacetic acid (EDTA). As shown herein, the hybrid nTP process can reliably transfer nanoparticle patterns (e.g., arrays of metal nanodots and nanowires) created by lithographic techniques, e.g., NIL, to soft and flexible casting materials (e.g., alginate hydrogels). Moreover, pattern-specific cell migration on the NIL-array printed casts and optimized casting material dissolution with EDTA maintained high cell viability. After dissociating the casting material (e.g., alginate hydrogel) transfer layer, the patterns bond to individual fibroblast cells. Advantageously, the hybrid nTP process offers a versatile strategy for seamless, tattooesque integration of patterns and arrays with live cells and tissues, while preserving viability. Further, the hybrid nTP process ensures substantial adherence of the patterns/arrays on the living cells, tissues, and soft aqueous and biological materials.

In a first aspect, a method of hybrid micro or nanotransfer printing (nTP) a nanopattern onto an article is described, said method comprising:
preparing the nanopattern comprising at least one nanopattern material on a first substrate;
transferring the nanopattern material onto a second substrate;
functionalizing the nanopattern material on the second substrate with at least one functionalizing compound;
casting the functionalized nanopattern material with at least one casting material to effectuate delamination of the nanopattern material from the second substrate;
chemically conjugating the nanopattern material with at least one conjugation compound to assist transfer of the nanopattern material onto the article;
positioning the chemically conjugated nanopattern material on the article; and
dissociating of the at least one casting material with a dissociation composition to yield the article comprising the nanopattern.

In some embodiments, the article is non-rigid. In some embodiments, the non-rigid article comprises cells, tissues, organs, and/or microorganisms. In some other embodiments, the article is rigid. In some embodiments, the article is living. In some embodiments, the article is wet, comprising an aqueous layer on at least a portion of the article. Advantageously, the method of the first aspect can be performed without killing the article.

In some embodiments of the first aspect, a method of hybrid micro or nanotransfer printing (nTP) a nanopattern onto a living article is described, said method comprising:
preparing the nanopattern comprising at least one nanopattern material on a first substrate;
transferring the nanopattern material onto a second substrate;
functionalizing the nanopattern material on the second substrate with at least one functionalizing compound;
casting the functionalized nanopattern material with at least one casting material to effectuate delamination of the nanopattern material from the second substrate;
chemically conjugating the nanopattern material with at least one conjugation compound to assist transfer of the nanopattern material onto the living article;
positioning the chemically conjugated nanopattern material on the living article; and
dissociating of the at least one casting material with a dissociation composition to yield the living article comprising the nanopattern.
   In some embodiments, the living article comprises cells, tissues, organs, and/or microorganisms. In some embodiments, the method can be performed without killing living the article.

In some embodiments, the nanopattern material comprises at least one element selected from the group consisting of Be, Mg, Ca, Sr, Ba, B, C, N, O, Al, Si, P, S, Ga, Ge, As, Se, Co, Ni, Cu, Zn, Ru, Rh, Pd, Ag, Cd, In, Sn, Sb, Te, Ti, Ta, Zr, W, Ir, Pt, Au, Hg, TI, Bi, combinations thereof, or alloys thereof. In some embodiments, the nanopattern material can consists substantially of a single element, e.g., C, Si, Ge, Sn S, Se, Te, or a metal such as gold or silver. In some embodiments, the nanopattern material comprises more than one element, e.g., SiOₓ. In some embodiments, the nanopattern material is mono-crystalline. In some embodiments, the nanopattern material is poly-crystalline. In some embodiments, the nanopattern material is amorphous. In some embodiments, the nanopattern material comprises a polymorph of an element (e.g., graphite, diamond, fullerene, carbon nanotube, graphene, graphyne). In some embodiments, the nanopattern material comprises at least two of mono-crystalline, poly-crystalline, and amorphous. In some embodiments, the nanopattern material comprises a group IV semiconductor compound (e.g., C, Si, Ge, Sn). In some embodiments, the nanopattern material comprises a group II-VI semiconductor compound (e.g., comprising at least one element from groups 2 (fka IIA) or 12 (fka IIB) with one element from group 16 (fka VIA) including, but not limited to, ZnO, ZnSe, ZnS, ZnTe, CdO, MgO, CdSe, CdS, CdTe). In some embodiments, the nanopattern material comprises a group III-V semiconductor compound (e.g., BN, AlN, GaN, InN, TlN, BP, AIP, GaP, InP, TlP, Bas, AlAs, GaAs, InAs, TlAs, BSb, AlSb, GaSb, InSb, TlSb, BBi, AlBi, GaBi, InBi, TlBi). In some embodiments, the nanopattern material comprises a high-K material (e.g., HfSiON, HfO₂, HfSiO). In some embodiments, the nanopattern material comprises an insulator (e.g., a high-dielectric constant material such as silicon nitride (SiN), tantalum oxide (Ta₂O₅), aluminum oxide (Al₂O₃), hafnium aluminum oxide (HfₓAl_{y}O), hafnium oxide (HfO₂), or titanium oxide (TiO₂).

In some embodiments, the first substrate comprises silicon, e.g., a silicon wafer.

In some embodiments, the process of preparing a nanopattern comprising at least one nanopattern material on the first substrate comprises:
coating a sacrificial layer onto the first substrate;
coating a resist layer onto the sacrificial layer;
lithographically transferring the nanopattern onto the resist layer;
removing residual resist from the nanopattern;
optionally depositing an adhesive layer on the nanopattern;
depositing the at least one nanopattern material on the nanopattern; and
removing the resist to produce the nanopattern comprising the nanopattern material on the first substrate.

In some embodiments, the coating of the sacrificial layer is performed by spin-coating, spray-coating or known vapor deposition techniques. In some embodiments, the sacrificial layer comprises polymethylglutarimide (PMGI) and is spin-coated onto the first substrate.

The material for the resist layer, and the method of removal of residual resist, is dependent on the lithographic process used. For example, if the lithographic process is nanoimprint lithography (NIL), the resist used is a resist specific for the NIL process, for example, it is able to be deformed by a stamp bearing the nanopattern. Following resist deformation, the residual resist is removed using plasma (e.g., O₂) etching or dissolution. If the lithographic process is photolithography, photolithographic masks bearing the nanopattern are used and the nanopattern is transferred to the resist using the mask and radiation, e.g., ultraviolet radiation. Following radiation exposure, the resist that was exposed to the radiation can be removed using, for example, dissolution or plasma etching. The person skilled in the lithographic arts is familiar with the various processes and materials required to transfer a pattern (e.g., a nanopattern) to a substrate. In some embodiments, the resist layer is deposited by spin-coating, spray-coating or known vapor deposition techniques. In some embodiments, the lithographic process used is NIL. In some other embodiments, the lithographic process used is photolithography. In some other embodiments, the lithographic process is e-beam lithography.

In some embodiments, following removal of the unwanted/residual resist, an adhesive layer is deposited onto the sacrificial layer prior to deposition of the at least one nanopattern material. In some embodiments, the nanopattern material is then deposited. Different deposition methods are known in the art, including thermal evaporation deposition, chemical vapor deposition (CVD), and physical vapor deposition (PVD), which permit the substantially even deposition of a layer of the nanopattern material over the entire substrate (comprising the sacrificial layer and the nanopatterned resist and optionally the adhesive layer).

Following deposition of the nanopattern material, the resist is removed from the substrate. In some embodiments, the resist is removed using a resist dissolution composition, as understood by the person skilled in the art. In some embodiments, the resist dissolution composition comprises acetone. In some embodiments, following removal of the resist, the nanopattern material (also referred to as the NIL-array herein) is positioned over the sacrificial layer, which is positioned on the first substrate.

For proof of concept herein, the example discussed includes the transferring of a nanopattern comprising a single nanopattern material (e.g., Au). It should be appreciated by the person skilled in the art that many different layers of nanopattern materials can be “printed” or transferred onto the first substrate.

In some embodiments, the process of transferring the nanopattern material, i.e., the NIL-array, onto a second substrate comprises:
coating a carrier film onto the first substrate comprising the nanopattern material, optional adhesive layer, and the sacrificial layer;
dissolving the sacrificial layer and removing the first substrate, yielding a carrier film comprising the nanopattern material;
positioning the carrier film comprising the nanopattern material on a second substrate; and
removing the carrier film such that the nanopattern material is positioned on the second substrate.

In some embodiments, the carrier film comprises polymethyl methacrylate (PMMA). In some embodiments, the carrier film is spin-coated onto the first substrate comprising the nanopattern material and the sacrificial layer. In some embodiments, the carrier film is spray-coated or deposited using known vapor deposition techniques. In some embodiments, the carrier film substantially conforms to the nanopattern material, regardless of how high the aspect ratio of the nanopattern material is. In some embodiments, the deposited carrier film is substantially thicker than the nanopattern material so as to maintain the integrity of the nanopattern material once released from the sacrificial layer. In some embodiments, if an adhesive layer was used, the adhesive layer is removed contemporaneously with the carrier film.

Following release of the carrier film comprising the nanopattern material from the sacrificial layer, the carrier film comprising the nanopattern material is positioned on a second substrate. The choice of the second substrate is dependent on the nanopattern material, wherein the second substrate preferably enables the efficient transfer of the nanopattern material to a casting material in a subsequent step. In some embodiments, the nanopattern material has relatively poor adhesion to the second substrate. In some embodiments, the second substrate comprises silicon, e.g., glass.

In some embodiments, following positioning of the nanopattern material on the second substrate, the carrier film is removed, for example, using dissolution or plasma etching. Following removal of the carrier film, the at least one nanopattern material is positioned directly on the second substrate.

In some embodiments, a first side of the nanopattern material on the second substrate is functionalized using at least one functionalizing compound. In some embodiments, the functionalizing compound comprises an amine (e.g., cysteamine, 3-Amino-1-propanethiol hydrochloride, 6-Amino-1-hexanethiol hydrochloride, 8-Amino-1-octanethiol hydrochloride, 11-Amino-1-undecanethiol hydrochloride), a carbodiimide (e.g., 1-ethyl-3-(3-dimethylaminopropyl) carbodiimide hydrochloride), a succinimide (e.g., N-hydroxysulfosuccinimide, N-hydroxysuccinimide), or any combination thereof. In some embodiments, the at least one functionalizing compound comprises an amine. In some embodiment, the at least one functionalizing compound comprises cysteamine. In some embodiments, the functionalizing compound (e.g., an amine) forms a monolayer on a first side of the nanopattern material.

In some embodiments, the functionalized nanopattern material is cast with at least one casting material to assist with the delamination of the nanopattern material from the second substrate. In some embodiments, the casting material comprises at least one of a hydrogel, a polymer, and/or a thin film. In some embodiments, the casting material comprises a hydrogel. In some embodiments, the hydrogel comprises an alginate hydrogel. In some embodiments, the casting material comprises a gelatin hydrogel. In some embodiments, the casting material substantially conforms to the functionalized nanopattern material, regardless of how high the aspect ratio of the nanopattern material is. In some embodiments, the casting material is substantially thicker than the nanopattern material so as to maintain the integrity of the nanopattern material once released from the second substrate.

After the casting material has been delaminated from the second substrate, the second side of the nanopattern material is chemically conjugated with at least one conjugation compound to assist transfer of the nanopattern material onto the article. In some embodiments, the chemical conjugation of the nanopattern material with the at least one conjugation compound comprises: functionalizing the second side of the nanopattern material with at least one functionalizing compound; and binding conjugation compound molecules to the functionalized nanopattern material. In some embodiments, the functionalizing compound is an amine (e.g., cysteamine, 3-Amino-1-propanethiol hydrochloride, 6-Amino-1-hexanethiol hydrochloride, 8-Amino-1-octanethiol hydrochloride, 11-Amino-1-undecanethiol hydrochloride), a carbodiimide (e.g., 1-ethyl-3-(3-dimethylaminopropyl) carbodiimide hydrochloride), a succinimide (e.g., N-hydroxysulfosuccinimide, N-hydroxysuccinimide), or any combination thereof. In some embodiments, the at least one functionalizing compound comprises an amine. In some embodiments, the at least one functionalizing compound comprises cysteamine. In some embodiments, the at least one functionalizing compound forms a monolayer on a second side of the nanopattern material. In some embodiments, the at least one functionalizing compound used to functionalize the second side of the nanopattern material is the same as, or different from, the at least one functionalizing compound used to functionalize the first side of the nanopattern material. The at least one conjugation compound permits the attachment of the nanopattern material to an article, e.g., cells, tissues, organs, etc. In some embodiments, the at least one conjugation compound comprises at least one of gelatin; adhesive proteins such as fibronectin, laminin, collagen, and derivatives thereof, and any combination thereof. In some embodiments, the at least one conjugation compound comprises gelatin. In some embodiments, the at least one conjugation compound comprises collagen. In some embodiments, the article conformally contacts the chemically conjugated nanopattern material.

Following attachment of the article to the chemically conjugated nanopattern material, in some embodiments, the casting material, e.g., hydrogel, comprising the article is positioned on a third substrate such that the article is positioned between the third substrate and the chemically conjugated nanopattern material. In some embodiments, the third substrate is coated with at least one conjugation compound. In some embodiments, the at least one conjugation compound coated on the third substrate is the same as, or different from, the at least one conjugation compound coated on the second side of the nanopattern material. In some embodiments, the third substrate is coated with gelatin. Following dissociation of the casting material with a dissociation composition, the article positioned on the third substrate comprises the nanopattern material. In some embodiments, the dissociation composition comprises ethylenediamine tetraacetic acid (EDTA), sodium citrate, alginate lyases, citric acid, hydrogen peroxide, and any combination thereof. In some embodiments, the dissociation composition comprises EDTA.

In some embodiments, the hybrid nTP method described herein does not include the use of at least one of organic solvents (e.g., toluene, acetone), high pressure (e.g., 3 bar), or high temperatures (e.g., 45-100° C.), or any combination thereof, once organic or living articles are added to the chemically conjugated nanopattern materials, as understood by the person skilled in the art.

It should be appreciated by the person skilled in the art that the nanopatterns may comprise arrays of specific structures, but in practice an array comprising 2 or more specific nanopatterns can be transferred from a first substrate to a second substrate, and ultimately cast with the casting material so that multiple different nanopatterns can be seeded or loaded with different articles, as understood by the person skilled in the art.

In a second aspect, an article comprising the nanopattern comprising the nanopattern material, as positioned on the article using the hybrid nTP process of the first aspect, is described herein. In some embodiments, the nanopattern conformally contacts the article. In some embodiments, the article comprises at least one of cells, tissues, organs, and microorganisms. In some embodiments, the article is alive. In some embodiments, the article remains viable following the positioning of the nanopattern onto the article.

As proof of concept, the hybrid nTP process can reliably transfer 8 mm×8 mm arrays of Au nanodots (250 nm diameter) and nanowires (300 nm width) created by NIL to soft and flexible alginate hydrogels. Pattern-specific cell migration on the Au NIL-array printed hydrogels was observed and alginate hydrogel dissolution with EDTA optimized to maintain high cell viability. After dissociating the alginate hydrogel transfer layer, it was observed that the Au NIL-arrays bonded to individual fibroblast cells. Advantageously, this hybrid nTP process described herein offers a versatile strategy for seamless, tattooesque integration of NIL-patterns and arrays with live cells and tissues.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner.

### Fabrication of the Au NIL-Arrays on Glass Coverslips

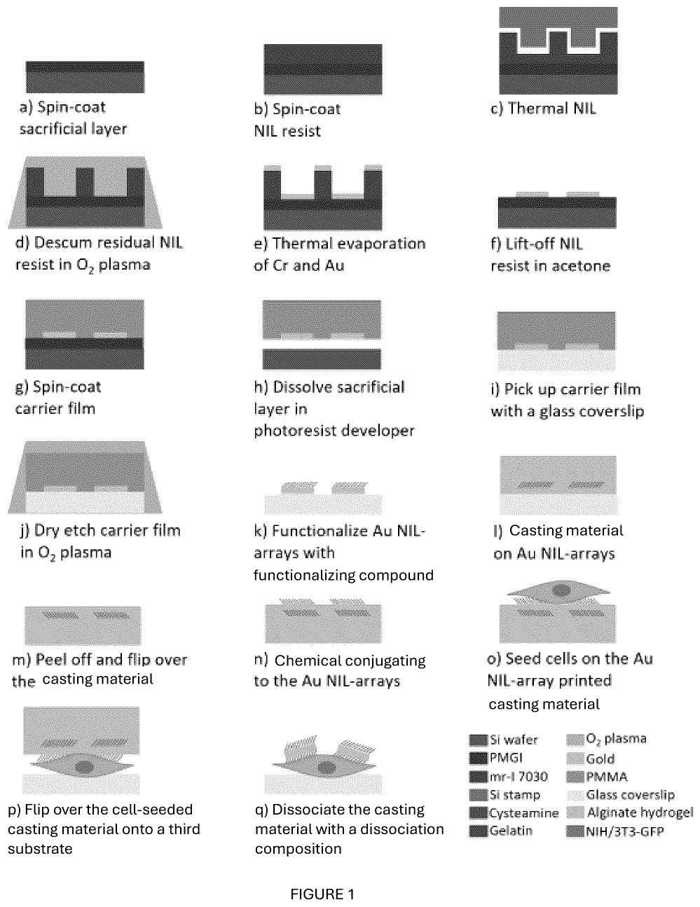

Au nanopattern arrays via nanoimprint lithography (NIL) were fabricated (see, FIG. 1). Briefly, a layer of polymethylglutarimide (PMGI SF6, Kayaku Advanced Materials) as the sacrificial layer was spin-coated on a silicon (Si) wafer (FIG. 1(a)). Then a layer of NIL resist (mr-I 7030, Micro Resist Technology) with a thickness of 350 nm was spin-coated (FIG. 1(b)) and thermally imprinted the nanopatterns using a Nanonex Advanced Nanoimprint Tool NX-B200 with a pressure of 350 psi at 130° C. A commercial, low-cost Si master stamp (LightSmyth grating) was used to create nanodots (approximately 250 nm diameter, 550 nm center-to-center spacing, 300 nm rim-to-rim spacing) and nanowires (approximately 300 nm width, 450 nm spacing) (FIG. 1(c)). After imprinting, the residual NIL resist was etched away (“descumed”) with oxygen plasma at 60 W for 2 minutes (FIG. 1(d)). Thermal evaporation was used to deposit 50 nm of Au, with a 5 nm thick Cr layer between the Au and the PMGI sacrificial layer to improve adhesion to the nanopatterned wafer (FIG. 1(e)). After deposition, the sample was sonicated in acetone to completely dissolve the NIL resist to obtain a large-area array (8 mm by 8 mm) of Au nanopatterns on the Si wafer (hereinafter the “Au NIL-array”) (FIG. 1(f)).

A layer of polymethyl methacrylate (950 PMMA A4) was spin-coated on top of the Au NIL-array as a carrier film (FIG. 1(g)). The Au NIL-array was released from the Si wafer by floating the Si wafer on top of a positive photoresist developer (MF-26A), dissolving the PMGI sacrificial layer (FIG. 1(h)). In some embodiments, the Si wafer is reused. To retain the shape of the nanopatterns, the thin film remained floating on the surface of the liquid. The film was rinsed with water by displacing the photoresist developer with deionized (DI) water three times. Then the Cr was etched in Cr etchant (Cr Cermet Etchant TFE, Transene) and the rinsing step was repeated with water. Then the film was picked up from the water-air interface using a glass coverslip (FIG. 1(i)). The choice of glass coverslips as the substrate enables efficient transfer of the Au NIL-arrays to the alginate hydrogel in the second step since Au has relatively poor adhesion to SiO₂ and oxide layers. After air-drying the film, the PMMA film was etched in oxygen plasma at 60 W for 30 minutes to obtain Au NIL-arrays on the glass coverslip (FIG. 1(j)). The NIL-arrays can be transferred onto glass coverslips with high fidelity, and it is noteworthy that such patterns can also be transferred onto rigid 3D shapes so long as the material poorly adheres to the material of the nanoparticle (e.g., the Au nanodots or nanowires).

### Alginate Hydrogel Preparation

Sodium alginate with high guluronic acid block content (average MW 177kDa, I1G, KIMICA) was used to fabricate the alginate hydrogel based on Chaudhuri. Briefly, alginate was purified by dialyzing it against DI water for 3 days with a 3500 MWCO membrane. Then activated charcoal and sterile filtration were used to purify the alginate. The purified alginate was lyophilized for 4-5 days and stored it at −20° C. until needed.

### Cell Culture

The NIH/3T3-GFP cells (kindly provided by Dr. Yun Chen at Johns Hopkins University) were cultured in standard DMEM (Gibco) with 10% fetal bovine serum (HyClone) and 1% penicillin/streptomycin (Gibco). The cells were cultured in a humidified incubator at 37° C. with 5% CO₂, kept at sub-confluency, and passaged every 2-3 days.

### Transfer of the Au NIL-Arrays to Cell Sheets and Rat Brains

The Au NIL-arrays were transferred from the glass coverslips onto cell sheets and tissues using alginate hydrogel as a biocompatible and sacrificial transfer layer. To facilitate the delamination of the Au NIL-array from the glass coverslip, the relative adhesion to the alginate hydrogel was enhanced by chemically modifying the Au surface with a self-assembled monolayer of cysteamine (FIG. 1(k)). The Au NIL-array was immersed in a 0.26 mM cysteamine ethanol solution for 1 hour. The alginate hydrogel was prepared by mixing 0.5 ml of the 2.5 wt % alginate solution with 125 μl of calcium sulfate to make the alginate hydrogel with a final calcium concentration of 25 mM. A homogenous mixture was obtained by loading each solution in a syringe and mixing with a dual Luer-lock connector. The alginate hydrogel was cast on the Au NIL-array and the solution was allowed to gel for 45 minutes under a glass slide with 1 mm-thick spacers (FIG. 1(l)). The alginate hydrogel containing the Au NIL-array was carefully peeled off from the glass coverslip and placed pattern-side up in a petri dish (FIG. 1(m)). The hydrogel was sterilized by placing it under UV light for an hour and immersed in excess CaCl₂ solution to prevent dehydration. Then the cysteamine functionalization step was repeated and the alginate hydrogel rinsed three times with DI water. To bind the gelatin molecules, the Au NIL-array with cysteamine functionalization was immersed in a 17.6 mM glutaraldehyde water solution for 30 minutes and the hydrogel rinsed three times with DI water. Next, the hydrogel was immersed in a 0.1% gelatin (Bloom 300, Type A) phosphate-buffered saline solution for an hour and the excess solution aspirated (FIG. 1(n)). The same gelatin coating procedure was used to obtain the gelatin-coated glass coverslips for a later step. After seeding NIH/3T3-GFP cells on the Au NIL-array printed alginate hydrogel (FIG. 1(o)), it was placed in an incubator for 24 hours. To obtain the Au NIL-array printed cells, the cell-seeded hydrogel was picked up and flipped it over onto a gelatin-coated coverslip so that the cells were in direct contact with the gelatin-coated coverslip (FIG. 1(p)). The cells were allowed to attach to the gelatin-coated coverslip overnight and the alginate hydrogel was dissociated by rinsing it with 20 mM of EDTA for about 9 minutes (FIG. 1(q)). For the transfer of the Au NIL-arrays to rat brains, the same alginate hydrogel casting and gelatin conjugation steps were repeated. Then the Au NIL-array printed alginate hydrogel was placed on top of the brain tissue so that the Au NIL-array was in direct contact with the tissue surface. After leaving the samples in cell culture media for about 2 hours, the alginate hydrogel was dissociated by rinsing it with 20 mM EDTA for about 9 minutes.

### Cell Tracking and Imaging

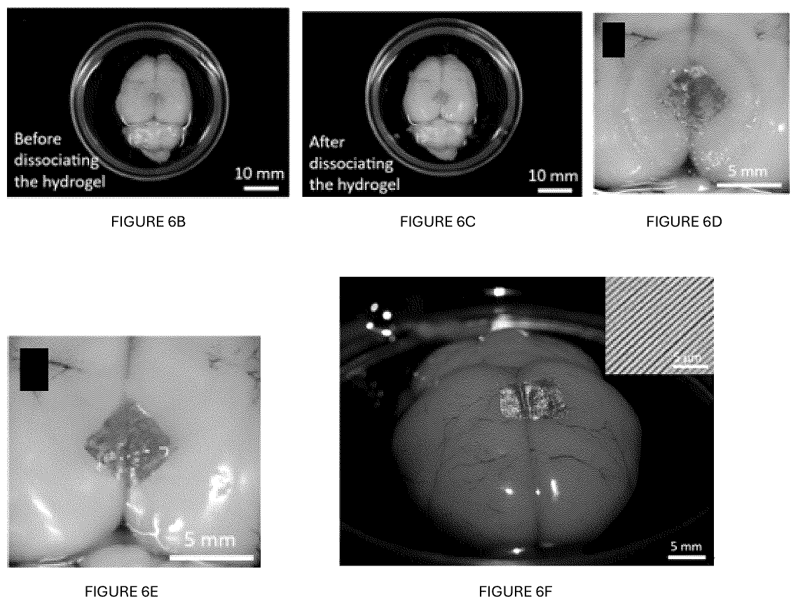

A Nikon TE2000 microscope with 10X objective lens was used to capture cell movement over 14 hours at 5-minute intervals. During imaging, cells were maintained on a temperature and CO₂-controlled stage in an incubator at 37° C. and 5% CO₂. CellTracker software was used to record cell migration paths and calculate cell migration speed. The inset in FIG. 6F was obtained using a Keyence laser scanning microscope VKX100.

### Animal Experiments

Sprague Dawley rats were purchased from Charles River and Taconic Biosciences, and the animals were bred and housed at Johns Hopkins animal facilities. All animal procedures and experiments were performed in accordance with guidelines set by the National Institutes of Health and the Johns Hopkins University Animal Care and Use Committee (ACUC). Postnatal 21-day rats were euthanized using carbon dioxide (CO₂). Rats were further subjected to cervical dislocation following euthanasia by CO₂ inhalation. Decapitation was performed, and brains were dissected for follow-up experiments.

### Results and Discussion

The transfer of the Au NIL-arrays to live cells and tissues requires certain criteria to be met, including flexibility, physical integrity, compatibility with cell culture media, and appropriately designed relative adhesion. Accordingly, specially designed hydrogels are an alternative to rigid substrates and can also act as a sacrificial layer by reverse gelation. Alginate is widely used for cell culture and tissue engineering due to its biocompatibility and tunable, tissue-mimetic mechanical properties. Therefore, an alginate hydrogel was selected as an intermediary substrate to delaminate the Au NIL-arrays from the rigid glass coverslip and affix them to cell sheets and brain tissues. In some embodiments, the adhesion between the Au NIL-array and the alginate hydrogel is greater than the adhesion between the Au NIL-array and the underlying substrate (e.g., glass, tissue, etc.) during the hydrogel assisted transfer of the Au NIL-arrays from the substrate to the alginate hydrogel. In some other embodiments, the adhesion between the Au NIL-array and the alginate hydrogel is substantially greater than the adhesion between the Au NIL-array and the underlying substrate (e.g., glass, tissue, etc.) during the during the hydrogel assisted transfer of the Au NIL-arrays from the substrate to the alginate hydrogel.

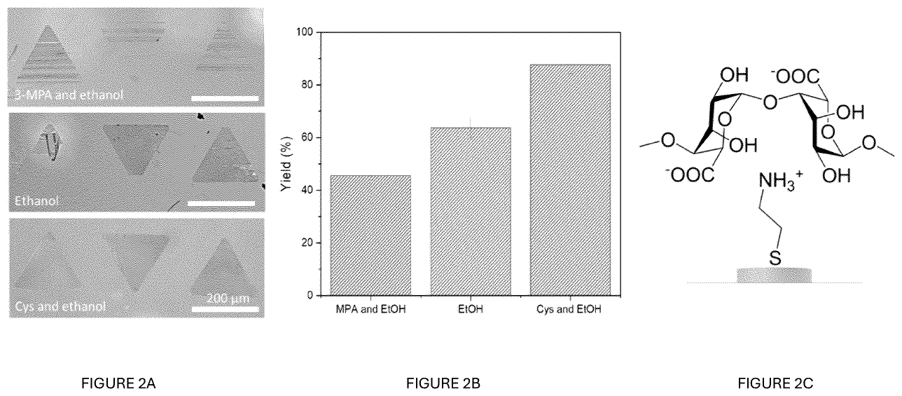

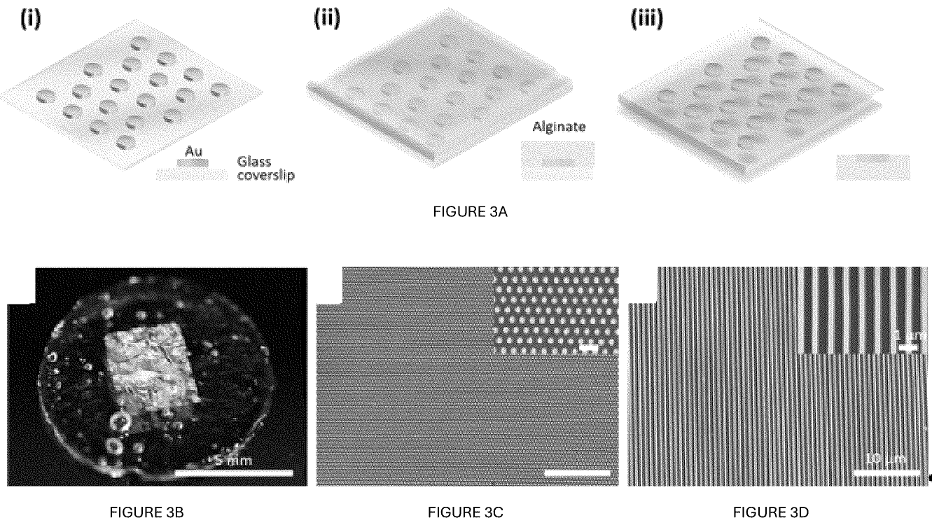

The effect of surface functionalization of Au on the adhesion of the Au NIL-array to the alginate hydrogel was investigated using self-assembled monolayers of either 3-mercaptopropionic acid (3-MPA) or cysteamine. Both molecules have a thiol group that can covalently bind to Au, but 3-MPA contains a negatively charged carboxylic acid end group in its deprotonated form [Lesiak et al., 2021], while cysteamine contains a positively charged amine end group in its protonated form [Thomas et al., 1995; Atallah et al., 2020]. The Au NIL-arrays were immersed in ethanol solutions containing either 0.26 mM 3-MPA or 0.26 mM cysteamine for 1 h. Then 0.5 mL of 2.5 wt % alginate solution was intermixed with 125 μL of calcium sulfate to make the alginate hydrogel with a final calcium concentration of 25 mM and cast the resulting alginate hydrogel on the Au NIL-arrays. After allowing the solution to gel for 45 min under a glass slide, the alginate hydrogel containing the Au NIL-array was gently peeled off from the glass coverslip and placed it pattern-side up for further characterization (see, FIG. 1). The transfer yield of the cysteamine-functionalized Au NIL-array was approximately twice that of the 3-MPA-functionalized Au NIL-array (FIG. 2). Without being bound by theory, this difference is attributed to favorable electrostatic forces between the positively charged end groups of the cysteamine molecules and the negatively charged carboxyl groups of alginate. Additionally, the SEM images (see, FIG. 3C and 3D) show that this transfer process can print both NIL-dots and NIL-wires with high fidelity.

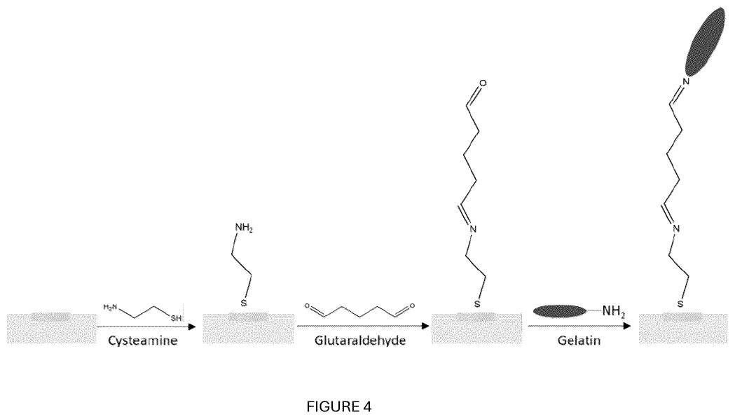

The viability, migration, and morphology of embryonic mouse fibroblast cells (NIH/3T3-GFP) on two different Au nanopatterns was investigated: dots (approximately 250 nm diameter, 550 nm center-to-center spacing, and 300 nm rim-to-rim spacing) and wires (approximately 300 nm wide, 450 nm spacing). As mammalian cells are known to have poor adhesion to alginate hydrogels, gelatin was bioconjugated to the Au NIL-array printed hydrogels prior to seeding NIH/3T3-GFP cells. The bioconjugation process (FIG. 4) involves sequential functionalization of the Au surface with cysteamine and glutaraldehyde and subsequent coating with 0.1% gelatin (Bloom 300, Type A). Glutaraldehyde contains aldehyde groups at both ends of the molecule and is known to bind to the amine groups in cysteamine and gelatin.

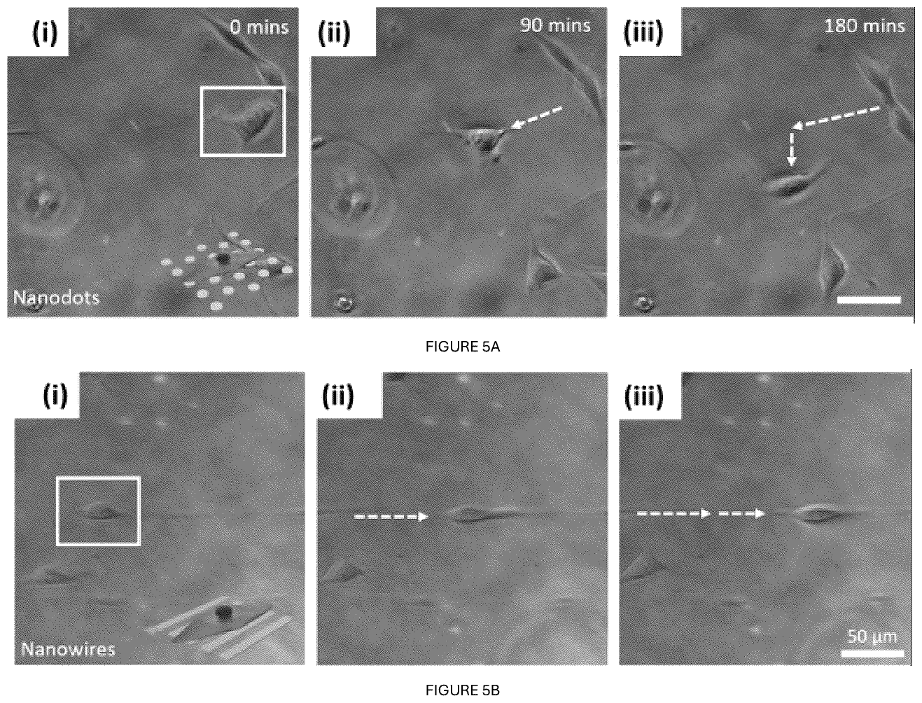

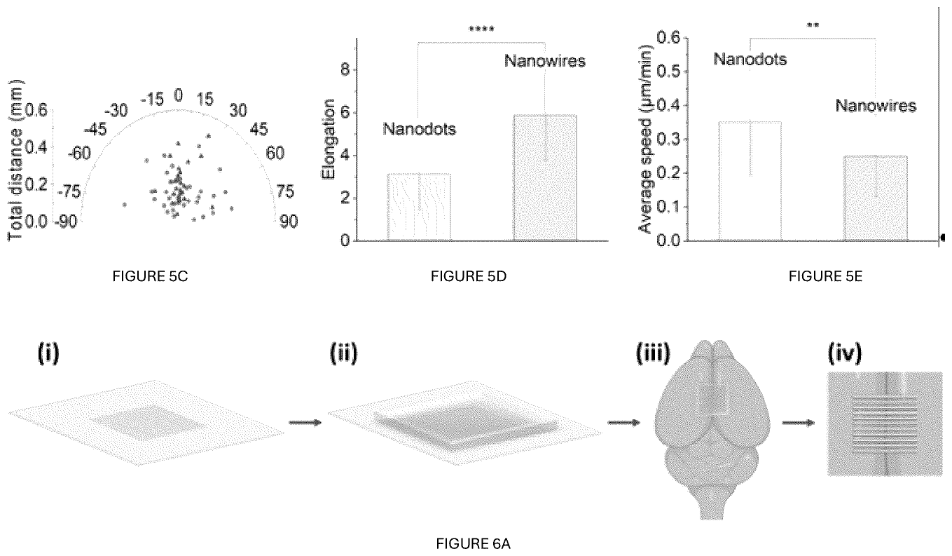

Of note, certain studies have reported that glutaraldehyde can undergo acetalization with the hydroxyl groups of alginate, while some have reported cross-linking reactions under acidic conditions. Conversely, glutaraldehyde exhibits rapid reactivity with amine groups and forms thermally and chemically stable cross-links around neutral pH. Therefore, the reaction conditions of the method described herein, including pH, concentration, temperature, and reaction times, must be optimized to achieve the desired cross-linking of glutaraldehyde with cysteamine and gelatin, instead of alginate. Approximately 24 h after seeding the cells on the Au NIL-array printed hydrogels, it was observed that the NIH/3T3-GFP cells on the NIL-wire printed hydrogel preferably migrated parallel to the nanowires (FIGS. 5B-5C), whereas those on NIL-dots exhibited random migration (FIGS. 5A and 5C). Using ImageJ, the elongation factor of the fibroblasts on the Au NIL-array printed hydrogels was estimated by measuring the long axis length over the short axis length of the cell. The elongation factor of the cells on the Au NIL-wire printed hydrogel was approximately twice that of the cells on the Au NIL-dot printed hydrogel (FIG. 5D). This observation suggests that the gelatin selectively conjugated to the Au NIL-arrays, thus enhancing cell alignment and elongation on the Au NIL-wire printed alginate hydrogel compared to the Au NIL-dot printed alginate hydrogel. Also, cells on the Au NIL-dot printed hydrogel migrated about 1.4 times faster than cells on the Au NIL-wire printed hydrogel (FIG. 5E). Accordingly, the method described herein enables fabrication of soft hydrogel and physiologically relevant substrates with tunable and precisely engineered surface patterns for investigation of cell morphology and dynamics.

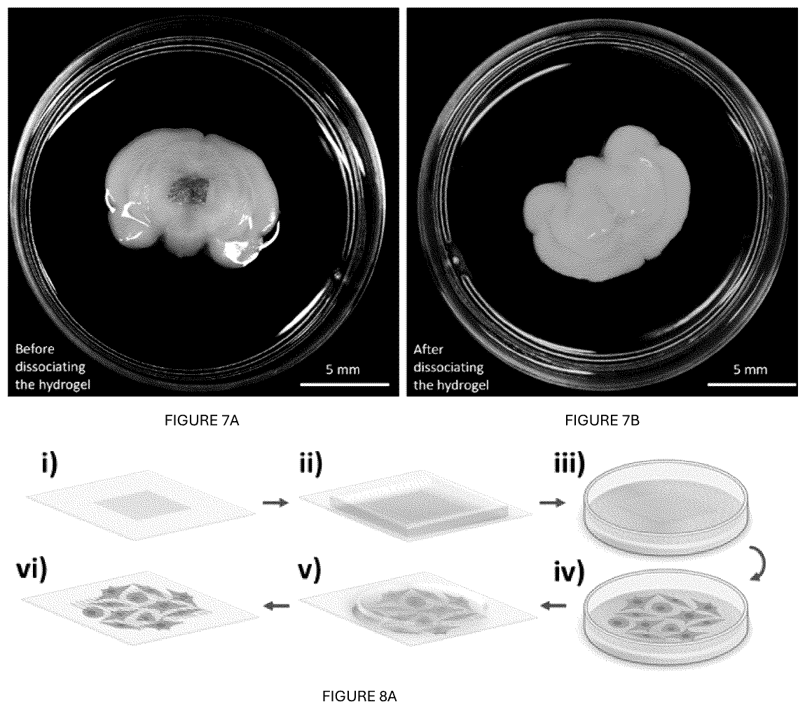

Alginate hydrogel not only is biocompatible with cells and tissues but can also undergo reverse gelation by metal chelates (e.g., EDTA) and specific enzymes (e.g., alginate lyases). Hence, it is an attractive sacrificial material for transferring Au NIL-arrays onto living organs and cells. To demonstrate, printed Au NIL-arrays were biotransferred on a whole rat brain (FIGS. 6B-6E) and on a rat brain slice (FIG. 7A). First, brains from 21-day postnatal rats were dissected and the Au NIL-wire printed alginate hydrogels positioned on the cerebral cortex of a whole brain and on a coronal brain slice. After leaving the samples in cell culture media for 2 h (FIG. 6A), the alginate hydrogels were dissociated with 20 mM EDTA. It was observed that the Au NIL-wires remained bonded to the surface of the whole brain (FIGS. 6D-6F). In contrast, the Au NIL-wires on the coronal brain slice (FIG. 7A) did not adhere and were washed away after rinsing with EDTA (FIG. 7B). Prior studies have shown that thin film patterns can conform to brain surfaces through physical adhesion forces like water capillarity or by interfacial hydrogel layers [Kim et al., 2010; Viventi et al., 2011; Oribe et al., 2019; Yang et al., 2021]. In the experiments described herein, the Au NIL-arrays selectively adhered to the surface of the whole rat brain, which exhibits cell and matrix compositions distinct from those of the coronal brain slice. Without being bound by theory, this suggests that the Au NIL-array adhesion mechanism may be cell-type-specific and cell-adhesion-related.

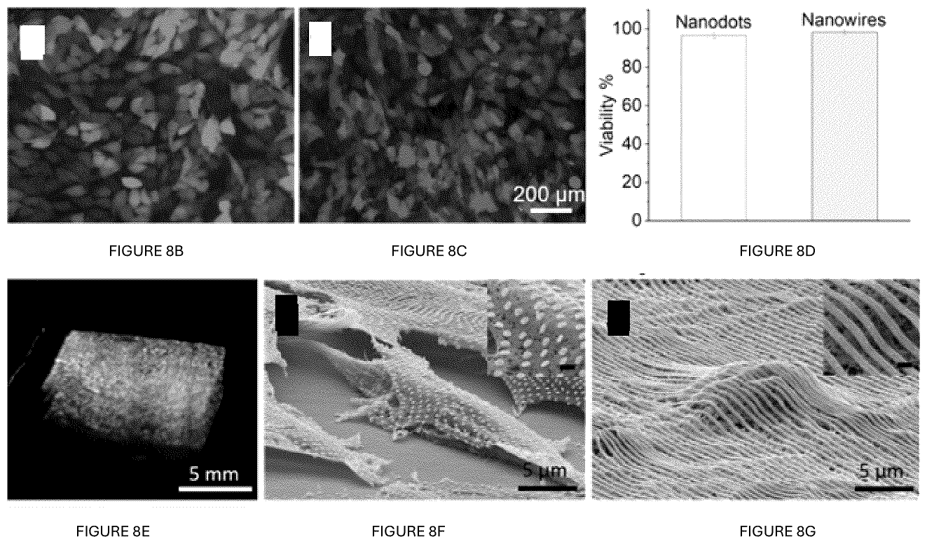

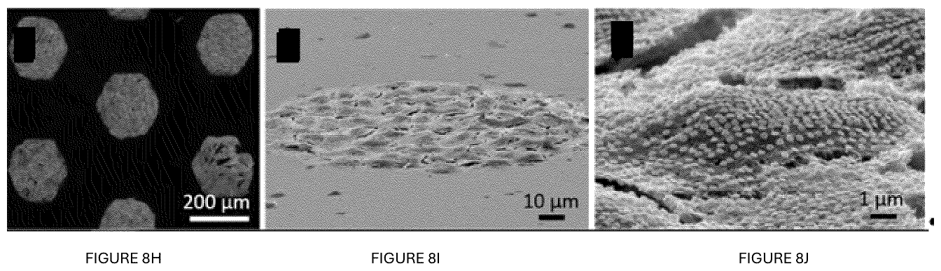

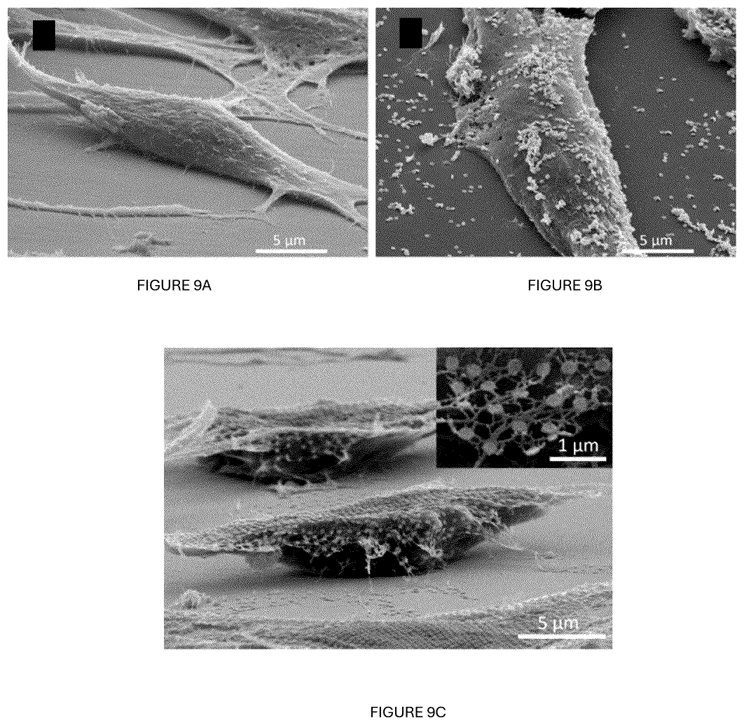

To assess the biotransfer printing capacity at the single-cell level, Au NIL-arrays were transferred onto a monolayer of fibroblasts. The biotransfer printing process demonstrated herein was inspired by cell sheet transfer with slight modifications (FIG. 8A). Briefly, NIH/3T3-GFP monolayer cell sheets were cultured on Au NIL-array printed alginate hydrogels for about 24 h. Then the cell-seeded hydrogels were flipped over onto gelatin-coated coverslips and the cells attached to the coverslips overnight. The alginate hydrogels were dissociated by rinsing them with 20 mM EDTA for about 9 min. After dissociating the alginate hydrogels, the viability of the NIH/3T3-GFP cell sheets with Au NIL-arrays were analyzed using propidium iodide (PI). Using fluorescence microscopy, a high cell viability was qualitatively observed with both Au NIL-dots and NIL-wires (FIGS. 8B-8C). Specifically, the fibroblasts patterned with Au NIL-dots had a viability of approximately 97%, while those patterned with Au NIL-wires had a viability of approximately 98% (FIG. 8D). These results indicate that this transfer printing process is biocompatible with live cells. Reflective colors were also observed from the fibroblast cell sheet patterned with the Au NIL-array (FIG. 8E), which suggests that the shape of the nanopattern array was retained on the cell sheet. The cell sheets patterned with Au NIL-arrays were fixed for SEM analysis and both the Au NIL-dots (FIG. 8F) and the NIL-wires (FIG. 8G) achieved conformal contact with the cell sheets. However, when the Au NIL-arrays were biotransfer-printed on cells cultured for shorter times, such as 4 h, the patterns were either distorted or fragmented (FIG. 9B). Concurrently, the presence of a thin, porous film on the cells cultured for at least 24 h was observed (FIG. 9C) but not on the cells cultured for a shorter period (FIGS. 9A-9B). Based on these SEM images, it is inferred that this thin, porous film represents extracellular matrix (ECM) secreted by NIH/3T3-GFP cells and that it may be involved in facilitating adhesion between the cell sheets and the Au NIL-arrays.

The fabrication process described herein is compatible not only with NIL but also with microscale photolithography (FIGS. 8H-8J). To illustrate this feature, photolithography and wet etching were used to define 200 μm wide hexagonal patches and 200 μm wide triangular patches of Au NIL-arrays. The micropatches of Au NIL-arrays were then biotransfer-printed onto cell sheets according to the steps shown in FIG. 8A. The bioconjugation of gelatin to the Au surface resulted in the selective growth of fibroblast cells on the micropatches of Au NIL-arrays, as the alginate hydrogel itself does not contain any cell-adhesion ligands necessary for promoting cell attachment (FIG. 8H). After dissociating the alginate hydrogel with EDTA, a high yield of Au NIL-array printed microcell patches attached to the coverslips were observed. The cells with Au NIL-wires appeared healthy and able to migrate, indicating biocompatibility of the transfer process. Further, the Au NIL-wires adhered and moved with cells during this period of migration (approximately 16 hours).

### Conclusion

In the present example, a biocompatible and cost-effective transfer process was presented that leverages (a) NIL to define sub-300 nm gold (Au) nanopattern arrays, (b) functionalization (e.g., with an amine) of Au to transfer the NIL-arrays from a rigid substrate to a soft transfer layer, (c) a flexible, degradable transfer casting layer (e.g., alginate hydrogel), and (d) chemical conjugation of the Au NIL-arrays (e.g., using gelatin) to achieve conformal contact with live cells. Biotransfer printing of the Au NIL-arrays on rat brains and live cells with high pattern fidelity and cell viability was demonstrated and differences in cell migration on the Au NIL-dot and NIL-wire printed hydrogels observed. It is anticipated that this nanolithography-compatible biotransfer printing method could advance bionics, biosensing, and biohybrid tissue interfaces and be used for new cell culture substrates.

In summary, a new approach for creating nanobio interfaces in the form of Au NIL-arrays on live cells and tissues has been disclosed. The approach utilizes molecular cross-linkers for the careful manipulation of adhesion between dissimilar materials and alginate as both a cell culture hydrogel substrate and degradable transfer layer. The ability of the Au NIL-arrays printed on physiologically relevant and ultrasoft substrates such as hydrogels to guide cell orientation and migration was demonstrated. By dissociating the alginate hydrogel with EDTA, conformal contact between the Au NIL-arrays and ex vivo rat brains as well as live cells was achieved. Importantly, NIL patterning enables facile integration of multifunctional devices in a high-throughput manner. Therefore, this approach could allow advanced functional optical and electronic devices, such as metamaterial arrays, plasmonic sensors, transistors, circuits, and antennas, to be imprinted on hydrogels, live cells, and tissues.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Ahn, J.; Ha, J.-H.; Jeong, Y.; Jung, Y.; Choi, J.; Gu, J.; Hwang, S. H.; Kang, M.; Ko, J.; Cho, S.; Han, H.; Kang, K.; Park, J.; Jeon, S.; Jeong, J.-H.; Park, I. Nanoscale Three-Dimensional Fabrication Based on Mechanically Guided Assembly. Nat. Commun. 2023, 14 (1), 1-10, DOI: 10.1038/s41467-023-36302-9
Atallah, C.; Charcosset, C.; Greige-Gerges, H. Challenges for cysteamine stabilization, quantification, and biological effects improvement. J. Pharm. Anal. 2020, 10 (6), 499-516, DOI: 10.1016/j.jpha.2020.03.007
Carlson, A.; Bowen, A. M.; Huang, Y.; Nuzzo, R. G.; Rogers, J. A. Transfer Printing Techniques for Materials Assembly and Micro/nanodevice Fabrication. Adv. Mater. 2012, 24 (39), 5284-5318, DOI: 10.1002/adma.201201386
Chaudhuri, O.; Gu, L.; Klumpers, D.; Darnell, M.; Bencherif, S. A.; Weaver, J. C.; Huebsch, N.; Lee, H.-P.; Lippens, E.; Duda, G. N.; Mooney, D. J. Hydrogels with Tunable Stress Relaxation Regulate Stem Cell Fate and Activity. Nat. Mater. 2016, 15 (3), 326-334.
Jeong, J. W.; Yang, S. R.; Hur, Y. H.; Kim, S. W.; Baek, K. M.; Yim, S.; Jang, H.-I.; Park, J. H.; Lee, S. Y.; Park, C.-O.; Jung, Y. S. High-Resolution Nanotransfer Printing Applicable to Diverse Surfaces via Interface-Targeted Adhesion Switching. Nat. Commun. 2014, 5 (1), 1-12, DOI: 10.1038/ncomms6387
Jeong, J. W.; Arnob, M. M. P.; Baek, K.-M.; Lee, S. Y.; Shih, W.-C.; Jung, Y. S. 3D Cross-Point Plasmonic Nanoarchitectures Containing Dense and Regular Hot Spots for Surface-Enhanced Raman Spectroscopy Analysis. Adv. Mater. 2016, 28 (39), 8695-8704, DOI: 10.1002/adma.201602603
Kim, D.-H.; Viventi, J.; Amsden, J. J.; Xiao, J.; Vigeland, L.; Kim, Y.-S.; Blanco, J. A.; Panilaitis, B.; Frechette, E. S.; Contreras, D.; Kaplan, D. L.; Omenetto, F. G.; Huang, Y.; Hwang, K.-C.; Zakin, M. R.; Litt, B.; Rogers, J. A. Dissolvable Films of Silk Fibroin for Ultrathin Conformal Bio-Integrated Electronics. Nat. Mater. 2010, 9 (6), 511-517, DOI: 10.1038/nmat2745
Ko, J.; Kang, H. J.; Ahn, J.; Zhao, Z.-J.; Jeong, Y.; Hwang, S. H.; Bok, M.; Jeon, S.; Gu, J.; Ha, J.-H.; Rho, J.; Jeong, J.-H.; Park, I. Biocompatible Nanotransfer Printing Based on Water Bridge Formation in Hyaluronic Acid and Its Application to Smart Contact Lenses. ACS Appl. Mater. Interfaces 2021, 13 (29), 35069-35078, DOI: 10.1021/acsami.1c06225
Lesiak, A.; Banski, M.; Halicka, K.; Cabaj, J.; Zak, A.; Podhorodecki, A. pH-dependent fluorescence of thiol-coated CdSe/CdS quantum dots in an aqueous phase. Nanotechnology 2021, 32 (7), 075705, DOI: 10.1088/1361-6528/abc4a0
Oribe, S.; Yoshida, S.; Kusama, S.; Osawa, S.-I.; Nakagawa, A.; Iwasaki, M.; Tominaga, T.; Nishizawa, M. Hydrogel-Based Organic Subdural Electrode with High Conformability to Brain Surface. Sci. Rep. 2019, 9 (1), 13379, DOI: 10.1038/s41598-019-49772-z
Thomas, R. C.; Houston, J. E.; Crooks, R. M.; Kim, T.; Michalske, T. A. Probing Adhesion Forces at the Molecular Scale. J. Am. Chem. Soc. 1995, 117, 3830-3834, DOI: 10.1021/ja00118a019
Viventi, J.; Kim, D.-H.; Vigeland, L.; Frechette, E. S.; Blanco, J. A.; Kim, Y.-S.; Avrin, A. E.; Tiruvadi, V. R.; Hwang, S.-W.; Vanleer, A. C.; Wulsin, D. F.; Davis, K.; Gelber, C. E.; Palmer, L.; Van der Spiegel, J.; Wu, J.; Xiao, J.; Huang, Y.; Contreras, D.; Rogers, J. A.; Litt, B. Flexible, Foldable, Actively Multiplexed, High-Density Electrode Array for Mapping Brain Activity in Vivo. Nat. Neurosci. 2011, 14 (12), 1599-1605, DOI: 10.1038/nn.2973
Yang, Q.; Hu, Z.; Rogers, J. A. Functional Hydrogel Interface Materials for Advanced Bioelectronic Devices. Acc. Mater. Res. 2021, 2 (11), 1010-1023, DOI: 10.1021/accountsmr.1c00142

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method of hybrid micro or nanotransfer printing (nTP) a nanopattern onto an article, said method comprising:
preparing the nanopattern comprising at least one nanopattern material on a first substrate;
transferring the nanopattern material onto a second substrate;
functionalizing the nanopattern material on the second substrate with at least one functionalizing compound;
casting the functionalized nanopattern material with at least one casting material to effectuate delamination of the nanopattern material from the second substrate;
chemically conjugating the nanopattern material with at least one conjugation compound to assist transfer of the nanopattern material onto the article;
positioning the chemically conjugated nanopattern material on the article; and
dissociating of the at least one casting material with a dissociation composition to yield the article comprising the nanopattern.

2. The method of claim 1, wherein the article comprises at least one of cells, tissues, organs, and microorganisms.

3. The method of claim 1, wherein the article is alive.

4. The method of claim 1, wherein the nanopattern material comprises at least one element selected from the group consisting of Be, Mg, Ca, Sr, Ba, B, C, N, O, Al, Si, P, S, Ga, Ge, As, Se, Co, Ni, Cu, Zn, Ru, Rh, Pd, Ag, Cd, In, Sn, Sb, Te, Ti, Ta, Zr, W, Ir, Pt, Au, Hg, TI, Bi, combinations thereof, or alloys thereof.

5. The method of claim 1, wherein the nanopattern material consists substantially of a single element.

6. The method of claim 1, wherein the nanopattern material comprises more than one element.

7. The method of claim 1, wherein the nanopattern material comprises at least one of a group IV semiconductor compound, a group II-VI semiconductor compound, a group III-V semiconductor compound, a high-K material, a metal, a metal alloy, an insulator, and any combination thereof.

8. The method of claim 1, wherein the nanopattern is in a form of a substantially spherical nanodot, a pyramidal particle, a multi-arm particle, a cubic nanoparticle, a nanotube particle, a nanowire particle, a nanofiber particle, a nanoplate particle, or any combination thereof.

9. The method of claim 1, wherein the nanopattern comprises gold.

10. The method of claim 1, wherein the preparing of a nanopattern comprising at least one nanopattern material on the first substrate comprises:
coating a sacrificial layer onto the first substrate;
coating a resist layer onto the sacrificial layer;
lithographically transferring the nanopattern onto the resist layer;
removing residual resist from the nanopattern;
optionally depositing an adhesive layer on the nanopattern;
depositing the at least one nanopattern material on the nanopattern; and
removing the resist to produce the nanopattern comprising the nanopattern material on the first substrate.

11. The method of claim 1, wherein the transferring of the nanopattern material onto a second substrate comprises:
coating a carrier film onto the first substrate comprising the nanopattern material, optional adhesive layer, and the sacrificial layer;
dissolving the sacrificial layer and removing the first substrate, yielding a carrier film comprising the nanopattern material;
positioning the carrier film comprising the nanopattern material on a second substrate; and
removing the carrier film such that the nanopattern material is positioned on the second substrate.

12. The method of claim 1, wherein a first side of the nanopattern material is functionalized using at least one functionalizing compound comprises an amine, a carbodiimide, a succinimide, or any combination thereof and forms a monolayer on the first side of the nanopattern material.

13. The method of claim 1, wherein the casting material comprises at least one of a hydrogel, a polymer, or a thin film.

14. The method of claim 1, wherein the chemical conjugation of the nanopattern material with at least one conjugation compound comprises:
functionalizing a second side of the nanopattern material with at least one functionalizing compound; and
binding the conjugation compound molecules to the functionalized nanopattern material.

15. The method of claim 14, wherein the at least one functionalizing compound comprises an amine, a carbodiimide, a succinimide, or any combination thereof and forms a monolayer on the second side of the nanopattern material.

16. The method of claim 1, wherein the at least one conjugation compound comprises at least one of gelatin, fibronectin, laminin, collagen, derivatives thereof, and any combination thereof.

17. The method of claim 1, wherein following the positioning of the chemically conjugated nanopattern material on the article, the casting material comprising the nanopattern material on the article is positioned on a third substrate such that the article is positioned between the third substrate and the chemically conjugated nanopattern material.

18. The method of claim 1, wherein the dissociation composition comprises ethylenediamine tetraacetic acid (EDTA), sodium citrate, alginate lyases, citric acid, hydrogen peroxide, and any combination thereof.

19. The method of claim 2, wherein the cells are prokaryotic, eukaryotic, or mammalian cells.

20. An article comprising a nanopattern positioned thereon, wherein said nanopattern is printed on the article using the method of hybrid micro or nanotransfer printing (nTP) of claim 1.
